(19) United States
(12) Patent Application Publication
(10) Pub. No.: US 2026/0327577 A1
(43) Pub. Date: Oct. 22, 2026

(54) USE OF RECOMBINANT HUMAN THIOREDOXIN (RHTRX)

(71) Applicant: Texas Tech University System, Lubbock, TX (US)

(72) Inventor: Kumuda Das, Lubbock, TX (US)

(73) Assignee: Texas Tech University System, Lubbock, TX (US)

(21) Appl. No.: 19/103,491

(22) PCT Filed: Aug. 15, 2023
(86) PCT No.: PCT/US2023/030204

§ 371 Date: Feb. 12, 2025

Related U.S. Application Data
(60) Provisional application No. 63/398,093, filed on Aug. 15, 2022.

(51) Int. Cl. C12N 9/02; A61K 38/00

(52) U.S. Cl. CPC C12N 9/0036; A61K 38/00; C12Y 108/01008

(57) ABSTRACT

Embodiments of the present disclosure pertain to a composition that is suitable for use in treating or preventing arterial stiffness in a subject. In some embodiments, the composition includes a recombinant human thioredoxin (rhTrx), derivatives thereof, peptide fragments thereof, mutants thereof, or combinations thereof. Additional embodiments of the present disclosure pertain to methods of treating or preventing arterial stiffness in a subject by administering the compositions of the present disclosure to the subject.

### CROSS-REFERENCE TO RELATED APPLICATIONS

This application claims priority to U.S. Provisional Patent Application No. 63/398,093, filed on Aug. 15, 2022. The entirety of the aforementioned application is incorporated herein by reference.

### STATEMENT REGARDING FEDERALLY SPONSORED RESEARCH

This invention was made with government support under R01 HL 132953 awarded by the National Institutes of Health. The government has certain rights in the invention.

### STATEMENT UNDER 37 C.F.R. § 1.834(C)(1)

Pursuant to 37 C.F.R. § 1.834, Applicant hereby submits a sequence listing as an XML file (“Sequence Listing”). The name of the file containing the Sequence Listing is “AF13368.P028WO.xml”. The date of the creation of the Sequence Listing is Aug. 15, 2023. The size of the Sequence Listing is 4,000 bytes. Applicant hereby incorporates by reference the material in the Sequence Listing.

### BACKGROUND

Arterial stiffness is an underlying mechanism for cardiovascular, cerebrovascular, and renal disorders, such as, for example, high blood pressure, dementia, or chronic kidney diseases. There are currently no drugs or treatments to reverse, decrease, or treat arterial stiffness. Various embodiments of the present disclosure seek to address these shortcomings.

### SUMMARY

In some embodiments, the present disclosure pertains to a composition that is suitable for use in treating or preventing arterial stiffness in a subject. In some embodiments, the composition includes a recombinant human thioredoxin (rhTrx), derivatives thereof, peptide fragments thereof, mutants thereof, or combinations thereof.

Additional embodiments of the present disclosure pertain to methods of treating or preventing arterial stiffness in a subject by administering the compositions of the present disclosure to the subject. In some embodiments, the arterial stiffness to be treated or prevented is associated with an arterial stiffness related disorder. In some embodiments, the arterial stiffness related disorder includes, without limitation, cardiovascular disorders, cerebrovascular disorders, renal disorders, high blood pressure, hypertension, dementia, chronic kidney disorders, chronic vascular fibrotic disorders, vascular fibrosis, cardiac fibrosis, inflammation, chronic low-grade inflammation in aging, and combinations thereof.

### DESCRIPTION OF THE DRAWINGS

FIG. 1 shows the mean arterial pressure of aged premenopausal female baboons (Papio anubis; n=3, Avg. age 26 years) that were injected with 2 dosages of rhTrx (125 μg/Kg) at 48 h intervals. The blood pressures of the baboons were continuously measured in conscious animals by radiotelemetry using an M11 catheter inserted in the carotid artery, before and after rhTrx injection. *P>0.05 compared to BP before rhTrx inj.

FIGS. 2A-2E show experimental results demonstrating that decreased expression of Trx and Sod2 in old premenopausal baboons is rescued by intravenous rhTrx injections (IV). Young and aged baboons were injected with 125 ug/Kg rhTrx (2 dosages at 48 h week intervals), following which animal were sacrificed and mRNA and protein expression was analyzed. FIG. 2A shows mRNA analysis by RT-PCR. FIG. 2B shows the densitometry of the results in FIG. 2A. FIG. 2C shows a Western analysis of Trx, Sod2 and β-actin in lysates of baboon aortae. FIGS. 2D-2E shows densitometry of Trx and Sod2; n=3 in Each group; ANOVA, *p<0.05 vs young; p<0.05 vs young.

FIGS. 3A-3B provide experimental results demonstrating no change in Trx2 or Sod1 proteins in female baboons untreated or treated with rhTrx. Shown are expression of Trx2 (FIG. 3A) and Sod1 (FIG. 3B) in baboon aortae, as described in FIGS. 2A-2E (n=3).

FIGS. 4A-4E provide experimental results demonstrating that rhTrx injection to aged baboons decreased the expression of MMP2/9: rhTrx injections. Baboons were treated with rhTrx as in FIG. 3A-3B. FIG. 4A shows mRNA levels, as determined by RT-PCR. FIG. 4B shows aortae lysates analyzed for MMP2/9 and β-actin. FIGS. 4C-4D show densitometry measurements (n=3) in each group; ANOVA, *p<0.05 vs young; **p<0.05 vs aged. FIG. 4E shows an MMP2-activity assay.

FIGS. 5A-5B provide experimental results demonstrating that decreased expression of Gpx4 in arteries is rescued by rhTrx injections. Young and Aged baboons were injected with 125 ug/Kg rhTrx (2 dosages at 48 h intervals), following which animal were sacrificed and protein expression was analyzed. FIG. 5A shows western analysis of Gpx4 and 3-actin in lysates of baboon aortae. FIG. 5B shows densitometry measurements of FIG. 5A. Young (N=3), Aged (N=4), N=5, Young+Trx (N=5), Aged+Trx (N=4); ANOVA, *p<0.05 vs young; **p<0.05 vs Aged.

FIGS. 6A-6C provide experimental results demonstrating decreased collagen I and increased elastin expression in the aortae of aged premenopausal female baboons. Young and Aged baboons were injected with 2.5 mg/Kg rhTrx (2 dosages at 2 week intervals), following which animals were sacrificed and protein expression was analyzed. FIG. 6A shows Western analysis of elastin, collagen I and β-actin in lysates of baboon aortae. FIGS. 6B and 6C show densitometries of FIG. 6A, n=3 in each group; ANOVA, *p<0.05 vs young; p<0.05 vs young. (n=3)

FIGS. 7A-7D show decreased expression of NLRP3 and Caspases in old premenopausal baboons is rescued by rhTrx injections. The baboons were treated as mentioned in FIGS. 2A-2E. FIG. 7A shows a Western analysis of an arterial lysate. FIG. 7B shows a densitometry of NLRP3. FIG. 7C shows a densitometry of caspase-1. FIG. 7D shows a densitometry of p20. (n=3) in each group; ANOVA, *p<0.05 vs young; p<0.05 vs young.

FIGS. 8A-8B provide experimental results demonstrating that arteries from aged baboons showed increased NLRC4 levels. However, baboons treated with rhTrx had significantly lower levels of NLRC4.

FIG. 9 shows experimental results demonstrating that Trx prevents TGFβ1-induced Col1A1 expression in HAoAF. Human aortic adventitial fibroblasts (HAoAF) were treated with 1 ng/mL TGFβ1 in the presence or absence of 2 μg/mL rhTrx for 16 hours. Cells were lysed and analyzed for Col1A1 by western blotting. N=3, **P<0.05; *P<0.05.

FIGS. 10A-10C provide experimental results demonstrating that Trx blocks TGFβ1-induced SMC α-actin expression and polymerization. HAoAF cells were treated with or without 1 ng/mL TGFβ1 in presence of 2 μg/mL rhTrx for 16 hours. As shown in FIG. 10A, Factin and α-SMC were stained by phalloidin-Alexa Fluor 568 and anti-SMC α-actin antibodies followed by Alexa Fluor 488-conjugated secondary antibodies. Shown are TGFβ without rhTrx (FIG. 10A—upper 2 panels), and TGFb with Trx (FIG. 10A—lower 2 panels). FIGS. 10B and 10C show F-actin (FIG. 10B) and a-SMC (FIG. 10C) levels. *, p<0.01 versus control; **, p<0.01 versus TGFβ1, ANOVA. (n=3).

FIG. 11 provides experimental results related to hydroxyproline (HP) levels in aorta of young and aged baboons treated with rhTrx. Arterial tissue was digested in HCL, and HP levels were determined by a colorimetric assay. N=3, *P=0.0229, Significantly higher than young; **P=0.0054, Significantly lower than aged baboons.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Arterial stiffness is an underlying mechanism for cardiovascular, cerebrovascular, and renal disorders, such as, but not limited to, high blood pressure, dementia, or chronic kidney diseases. Currently, there are no drugs or treatments to reverse or treat arterial stiffness. In addition, there are currently no treatments to decrease arterial stiffness.

Accordingly, a need exists for more effective compositions and methods for treating or preventing arterial stiffness. Various embodiments of the present disclosure address the aforementioned need.

In an embodiment, the present disclosure pertains to compositions for use in treating or preventing an arterial stiffness in a subject. In some embodiments, the compositions of the present disclosure include a recombinant human thioredoxin (rhTrx), derivatives thereof, peptide fragments thereof, or combinations thereof.

In an additional embodiment, the present disclosure pertains to a method of treating or preventing an arterial stiffness in a subject. Such methods generally include administering a composition of the present disclosure to the subject.

As outlined in further detail below, the compositions of the present disclosure can include numerous embodiments and be composed of various components and/or constituents. Additionally, the methods of the present disclosure can include various modes of administration to numerous subject types to treat or prevent a variety of arterials stiffness related disorders.

### Compositions

As set forth in further detail herein, the compositions of the present disclosure can have numerous embodiments. For example, the compositions of the present disclosure can include, without limitation, a recombinant human thioredoxin (rhTrx), derivatives thereof, peptide fragments thereof, or combinations thereof.

In some embodiments, the compositions of the present disclosure include recombinant human thioredoxin (rhTrx). In some embodiments, rhTrx includes SEQ ID NO: 1 (i.e., MVKQIESKTAFQEALDAAGDKLVVVDFSATWCGPCKMIKPFFHSLSEKYSNVIFLEVDV DDCQDVASECEVKCMPTFQFFKKGQKVGEFSGANKEKLEATINELV). In some embodiments, rhTrx includes a derivative or mutant of rhTrx. In some embodiments, the rhTrx derivative or mutant shares at least 95% sequence identity with SEQ ID NO: 1. In some embodiments, the rhTrx derivative or mutant shares at least 90% sequence identity with SEQ ID NO: 1. In some embodiments, the rhTrx derivative or mutant shares at least 85% sequence identity with SEQ ID NO: 1. In some embodiments, the rhTrx derivative or mutant shares at least 80% sequence identity with SEQ ID NO: 1. In some embodiments, the rhTrx derivative or mutant shares at least 75% sequence identity with SEQ ID NO: 1. In some embodiments, the rhTrx derivative or mutant shares at least 70% sequence identity with SEQ ID NO: 1. In some embodiments, the rhTrx derivative or mutant shares at least 65% sequence identity with SEQ ID NO: 1.

In some embodiments, the compositions of the present disclosure can include peptide fragments of rhTrx. In some embodiments, the peptide fragments are similar to thioredoxin (Trx) active sites. In some embodiment, the peptide fragment includes SEQ ID NO: 2 (i.e., KLVVVDFSATWCGPCKMIKPFFHSLSEKYSNVIFLEVDVDDCQDVASECEVKCMPTFQFF). In some embodiments, the peptide fragment includes a sequence that shares at least 65% sequence identity with SEQ ID NO: 2. In some embodiments, the peptide fragment includes a sequence that shares at least 70% sequence identity with SEQ ID NO: 2. In some embodiments, the peptide fragment includes a sequence that shares at least 75% sequence identity with SEQ ID NO: 2. In some embodiments, the peptide fragment includes a sequence that shares at least 80% sequence identity with SEQ ID NO: 2. In some embodiments, the peptide fragment includes a sequence that shares at least 85% sequence identity with SEQ ID NO: 2. In some embodiments, the peptide fragment includes a sequence that shares at least 90% sequence identity with SEQ ID NO: 2. In some embodiments, the peptide fragment includes a sequence that shares at least 95% sequence identity with SEQ ID NO: 2.

In some embodiment, the peptide fragment includes SEQ ID NO: 3 (i.e., KLVVVDFSATWCGPCKMIKPFFHSLSEKYSNVIFLEVDVDDAQDVASEAEVKAMPTFQFF). In some embodiments, the peptide fragment includes a sequence that shares at least 65% sequence identity with SEQ ID NO: 3. In some embodiments, the peptide fragment includes a sequence that shares at least 70% sequence identity with SEQ ID NO: 3. In some embodiments, the peptide fragment includes a sequence that shares at least 75% sequence identity with SEQ ID NO: 3. In some embodiments, the peptide fragment includes a sequence that shares at least 80% sequence identity with SEQ ID NO: 3. In some embodiments, the peptide fragment includes a sequence that shares at least 85% sequence identity with SEQ ID NO: 3. In some embodiments, the peptide fragment includes a sequence that shares at least 90% sequence identity with SEQ ID NO: 3. In some embodiments, the peptide fragment includes a sequence that shares at least 95% sequence identity with SEQ ID NO: 3.

In some embodiments, the compositions of the present disclosure may be formulated for administration in one or more doses. In some embodiments, the compositions of the present disclosure also include one or more stabilizers. In some embodiments, the stabilizers include, without limitation, anti-oxidants, sequestrants, ultraviolet stabilizers, or combinations thereof.

In some embodiments, the compositions of the present disclosure also include one or more surfactants. In some embodiments, the surfactants include, without limitation, anionic surfactants, sugars, cationic surfactants, zwitterionic surfactants, non-ionic surfactants, or combinations thereof.

In some embodiments, the compositions of the present disclosure also include one or more excipients. In some embodiments, the excipients include, without limitation, lactose, sucrose, starch powder, cellulose esters of alkanoic acids, trehalose, cellulose alkyl esters, talc, stearic acid, magnesium stearate, magnesium oxide, sodium and calcium salts of phosphoric and sulfuric acids, gelatin, acacia gum, trehalose, sodium alginate, polyvinylpyrrolidone, polyvinyl alcohol, or combinations thereof.

In some embodiments, the compositions of the present disclosure include a delivery vehicle, such as a particle. In some embodiments, the particle includes, without limitation, lipid-based particles, carbon-based particles, metal-based particles, or combinations thereof. In some embodiments, the active agents of the present disclosure are encapsulated in the particles.

In some embodiments, the compositions of the present disclosure can include additional components. For example, in some embodiments, the composition can further include dithiothreitol (threo-1,4 Dimercapto-2,3-butanediol), ethylenediaminetetraacetic acid sodium, glycerol (1,2,3-Propanetriol), an ammonium carbonate buffered solution, and combinations thereof.

In some embodiments, the compositions of the present disclosure can have various pH levels. In some embodiments, the compositions have a pH of about 6.5. In some embodiments, the compositions have a pH of about 7. In some embodiments, the compositions have a pH of about 7.5. In some embodiments, the compositions have a pH in the range of about 6.5 to about 7.5.

The compositions of the present disclosure can have various concentrations of human recombinant thioredoxin. For instance, in some embodiments, the compositions of the present disclosure have at least 50 μM human recombinant thioredoxin. In some embodiments, the compositions of the present disclosure have at least 75 μM human recombinant thioredoxin. In some embodiments, the compositions of the present disclosure have at least 85 μM human recombinant thioredoxin. In some embodiments, the compositions of the present disclosure have at least from 50 μM to 500 μM of human recombinant thioredoxin. In some embodiments, the compositions of the present disclosure have at least from 80 μM to 500 μM of human recombinant thioredoxin. In some embodiments, the compositions of the present disclosure have at least from 250 μM to 500 μM of human recombinant thioredoxin.

In a particular embodiment, the compositions of the present disclosure include, for example: (a) at least 85 μM human recombinant thioredoxin (e.g., 85.47 μM human recombinant thioredoxin); (b) 1 μM dithiothreitol (threo-1,4 Dimercapto-2,3-butanediol); (c) 0.5 μM ethylenediaminetetraacetic acid sodium; (d) 2% glycerol (1,2,3-Propanetriol); and (e) 0.5 mM ammonium carbonate buffered solution. In some embodiments, the composition has a total volume of 500 μl. In some embodiment, the composition is administered at a dose of about 125 to about 250 μl per kg of body weight of a subject (i.e., containing 250 μM to 500 μM human recombinant thioredoxin).

### Method of Treatment

As set forth in further detail herein, the compositions and methods of the present disclosure can have numerous embodiments. For example, in some embodiments, the compositions of the present disclosure can be used for treating or preventing arterial stiffness in a subject. Such methods generally include administering a composition as outlined in detail herein to a subject.

### Administering

As detailed herein, the methods of the present disclosure can utilize various modes and/or methods of administration. For example, in some embodiments, the administering occurs by a method that can include, without limitation, intravenous administration, intramuscular administration, intradermal administration, intraperitoneal administration, subcutaneous administration, spray-based administration, aerosol-based administration, and combinations thereof. In some embodiments, the administration occurs via intravenous administration.

In some embodiments, the administration can occur at varying doses. For instance, in some embodiments, the compositions of the present disclosure can be administered at a dose of about 125 to about 250 μl per kg of body weight of the subject (containing 250 μM to 500 μM human recombinant thioredoxin).

### Subject

As set forth in further detail herein, the methods of the present disclosure can be utilized on a variety of subjects. For example, in some embodiments, the subject is suffering from arterial stiffness. In some embodiments, the subject is vulnerable to arterial stiffness. In some embodiments, the subjects are human. In some embodiments, the subjects are baboons. In some embodiments, the subjects are mice.

### Treatment or Prevention

As detailed herein, the methods of the present disclosure can treat or prevent arterial stiffness in various manners. For instance, in some embodiments, the methods of the present disclosure prevent arterial stiffness. In some embodiments, the methods of the present disclosure treat arterial stiffness. In some embodiments, the methods of the present disclosure reverse arterial stiffness. In some embodiments, the methods of the present disclosure reduce arterial stiffness.

In some embodiments, the methods of the present disclosure treat or prevent arterial stiffness through decreasing the level of proteins associated with increasing arterial stiffness. In some embodiments, the proteins associated with increasing arterial stiffness can include, without limitation, collagen-1, metalloproteinase-2, metalloproteinase-9, endothelin-1, caspase-1, NLRP3, VCAM-1, or combinations thereof.

In some embodiments, the methods of the present disclosure treat or prevent arterial stiffness through increasing the level of proteins associated with decreasing arterial stiffness. In some embodiments, proteins associated with decreasing arterial stiffness can include, without limitation, elastin, superoxide dismutase-2, Glutathione peroxidase-4, or combinations thereof.

### Arterial Stiffness

As detailed herein, the methods of the present disclosure can be utilized to treat or prevent numerous types of arterial stiffness. For instance, in some embodiments, the arterial stiffness is associated with an arterial stiffness related disorder. In some embodiments, the arterial stiffness related disorder can include, without limitation, cardiovascular disorders, cerebrovascular disorders, renal disorders, high blood pressure, hypertension, dementia, chronic kidney disorders, chronic vascular fibrotic disorders, vascular fibrosis, cardiac fibrosis, inflammation, chronic low-grade inflammation in aging, and combinations thereof. In some embodiments, the arterial stiffness related disorders include cardiovascular disorders.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Effects of rhTrx in Baboons and Humans

This Example provides experimental results related to the effects of rhTrx in baboons and humans. In particular, Applicant observed that rhTrx decreases high blood pressure and arterial stiffness in premenopausal aged female baboons. Additionally, Applicant's experimental results demonstrated the efficacy of rhTrx in reducing vascular fibrosis in humans.

### Example 1.1. RhTrx Chronically Decreases High Blood Pressure in Premenopausal Aged Female Baboons

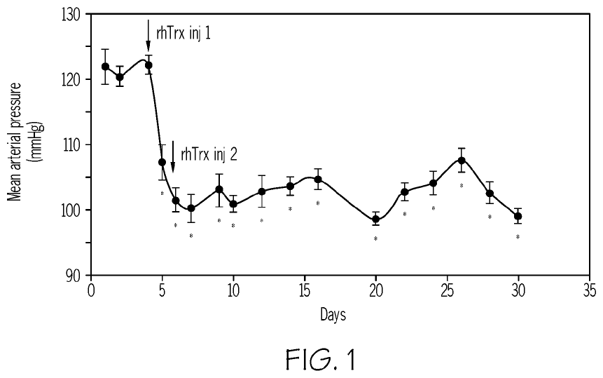

Applicant determined whether rhTrx injection would decrease high blood pressure in baboons. As shown in FIG. 1, a significant drop in blood pressure was achieved in the first injection, and the second injection maintained lower pressure for more than 30 days, indicating a more effective chronic decrease of blood pressure in baboons compared to mice.

Since, rhTrx induces Sod2 in nonhuman primates, but not in rodents, Applicant hypothesizes that rhTrx prevents mtROS mediated arterial ECM remodeling by chronically increasing expression of Sod2. Applicant next evaluated the level of Sod2 and Trx in the aged aortae and its modulation by rhTrx.

### Example 1.2. Decreased Trx and Sod2 mRNA and Protein Expression in Aortae of Aged Premenopausal Female Baboons are Rescued by Injection of rhTrx

Applicant previously showed that rhTrx induces Sod2 expression only in human or nonhuman primate cells, but not in rodents such as mice or rat. Therefore, Applicant sought to determine whether rhTrx would increase Sod2 expression in nonhuman primates.

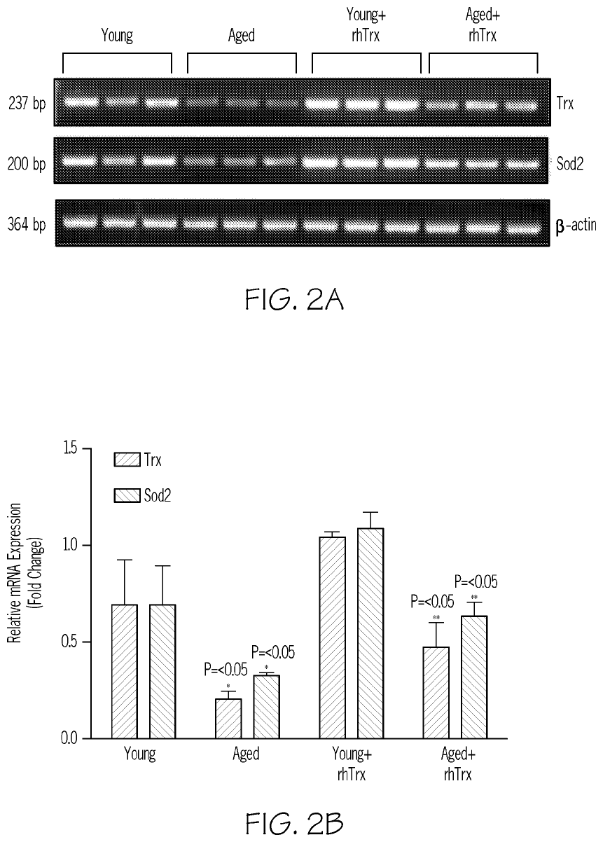

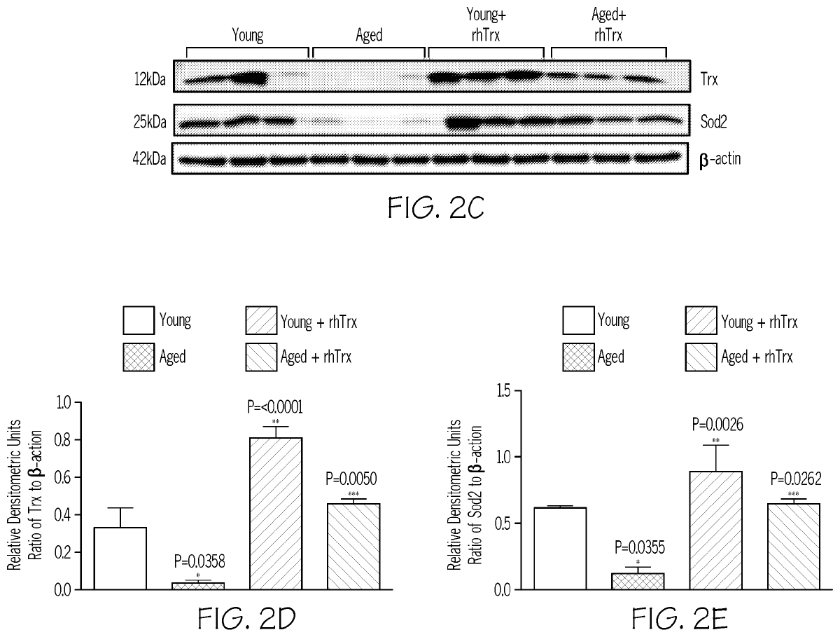

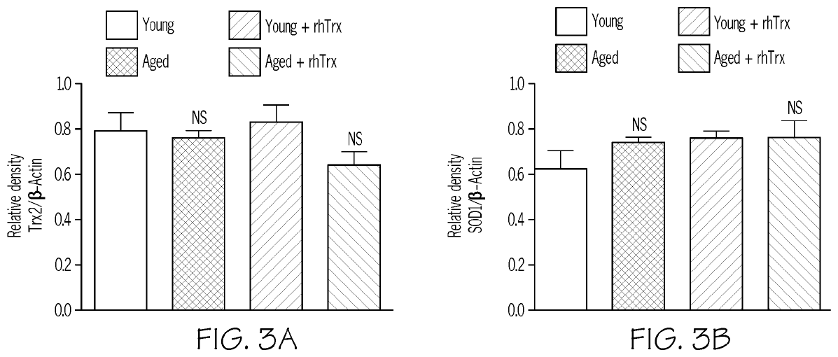

As shown in FIGS. 2A-2E, aged baboons showed very low levels of Sod2 and Trx mRNA and protein expression in their arteries. However, rhTrx administration induced significantly higher levels of Sod2 mRNA and protein in the aorta of aged and young female baboons, suggesting for the first time that rhTrx might have decreased mtROS due to increased Sod2 expression. In contrast to Trx, the level of Trx2 or Sod1 did not change in arteries of aged baboons (FIGS. 3A-3B) further suggesting a specific role of mtROS in arterial stiffness in aging.

### Example 1.3. Age-Related Increase in MMP2 and MMP9 mRNA and Protein Expression is Reduced in Premenopausal Female Baboons Treated with rhTrx

MMP2 and MMP9 have been shown to be associated with aging and are induced by proinflammatory mediators such as Ang II, ET-1, or cytokines. Activated MMPs regulate the synthesis of collagen, elastin and other ECM proteins resulting in vascular remodeling and stiffness. Applicant therefore evaluated whether rhTrx treatment decreases the expression of MMPs.

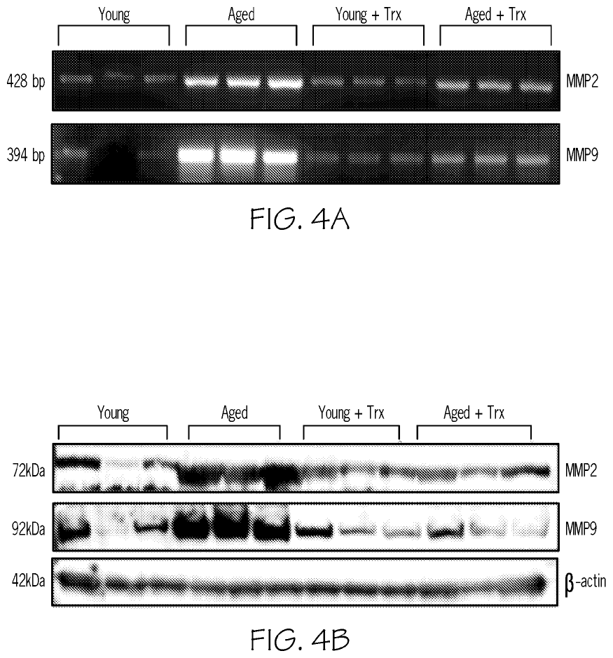

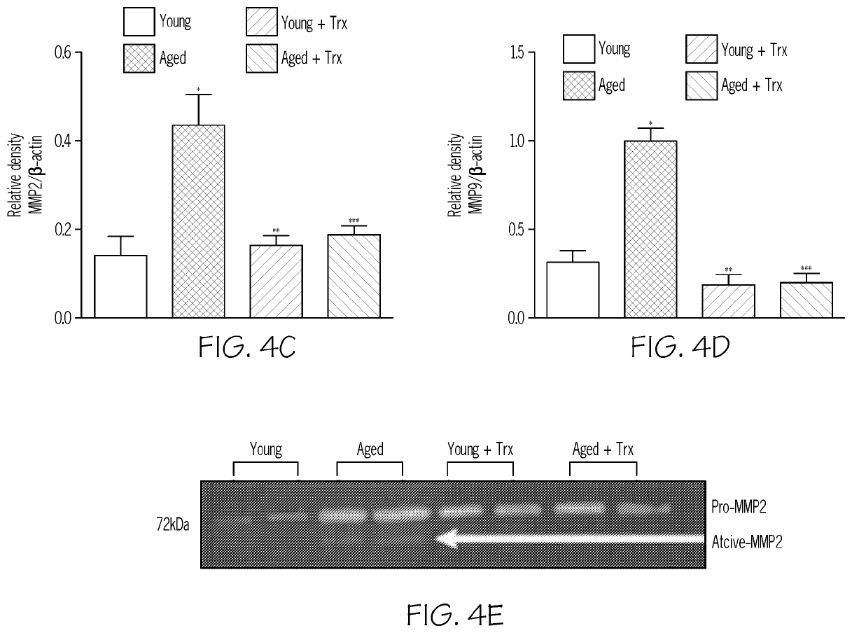

As shown in FIGS. 4A-4D, MMP mRNA and protein were induced in aged baboons, but were significantly decreased in baboons treated with rhTrx. Additionally, MMP2 activity was increased in aged baboons (FIG. 4E), which was decreased with rhTrx treatment. These data suggest that rhTrx would be effective in decreasing arterial stiffness by decreasing MMP expression and activation in aged baboon arteries.

### Example 1.4. The Expression of Gpx4 is Acutely Decreased in Arteries of Aged Baboons, but Treatment with rhTrx Rescues the Loss of Gpx4

The content of 8-Isoprostane and 4-HNE has been shown to be significantly increased in the arterial wall of aged monkeys compared with young vessels. The content of vascular GSH was also shown to be significantly decreased. Since Gpx4 is known to reduce lipid peroxidation products, Applicant evaluated whether aging decreases Gpx4 levels.

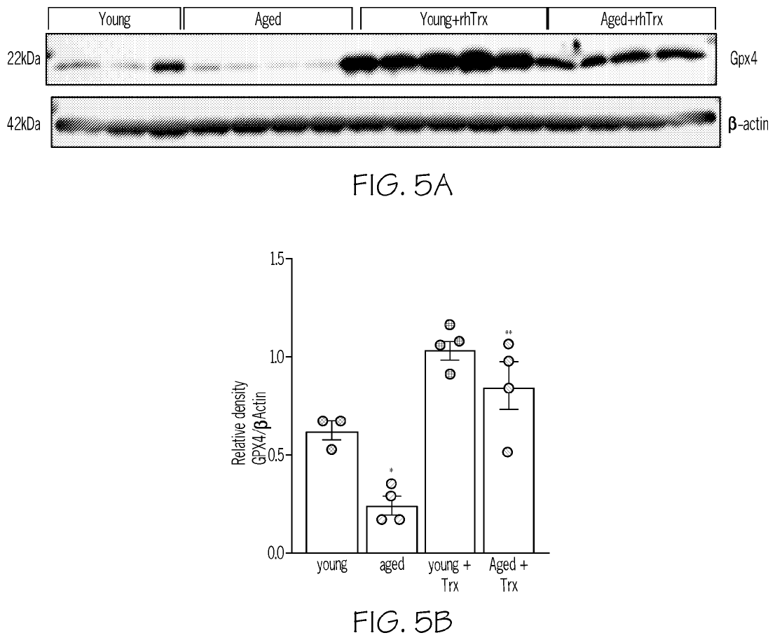

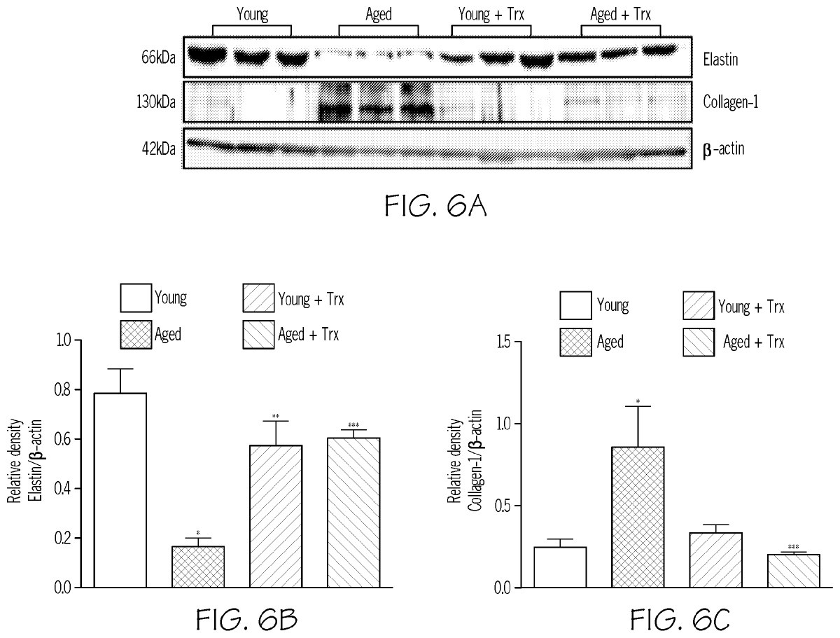

As shown in FIGS. 5A-5B, the expression of Gpx4 in the aorta was significantly decreased in aged baboons compared with young baboons. Moreover, rhTrx treatment of aged baboons rescued Gpx4 expression. This finding demonstrates that loss of Gpx4 may promote ferroptosis of vascular cells that accumulate lipid hydroperoxides in aging arteries, and rhTrx via Sod2 upregulation could have restored Gpx4 expression in aged baboons. Further, loss of VSMC by ferroptosis may activate fibroblast differentiation to myofibroblast as a repair mechanism.

### Example 1.5. RhTrx Treatment Decreased Collagen I and Increased Elastin Expression in the Aortae of Aged Premenopausal Female Baboons

Applicant determined the expression of major matrix proteins, collagen-I and elastin in aged and young female baboon aorta. As shown in FIGS. 6A-6C, the expression of elastin was significantly decreased, but the expression of collagen-1 was significantly increased in aged female baboons. In contrast, baboons injected with rhTrx showed increased elastin expression and decreased collagen-1 levels, suggesting rhTrx decreases arterial stiffness by improving elastin levels with decreased collagen deposition.

### Example 1.6. RhTrx Treatment Decreases Caspase-1, Caspase-1(p20) and NLRP3 Expression in Aged Female Baboons

Although chronic low-grade inflammation in aging (i.e., inflammaging) is a major cause of arterial stiffness, age-related mechanisms associated with inflammaging is unclear. Additionally, the mechanism of age-mediated increase in NLRP3 inflammasome activation remains poorly understood. Since mtROS is known to induce NLRP3 activation, Applicant reasoned that loss of Sod2 in aging would cause higher levels of mtROS that may trigger the NLRP3 activation.

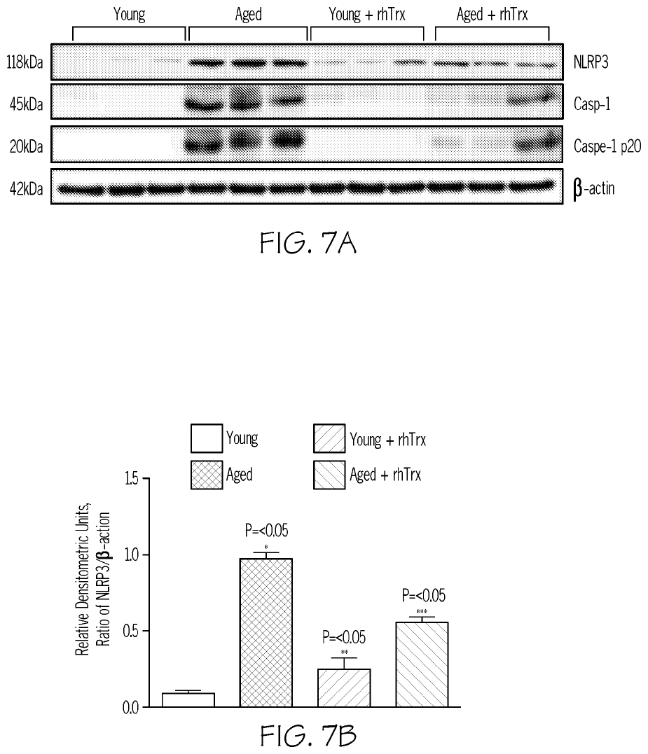

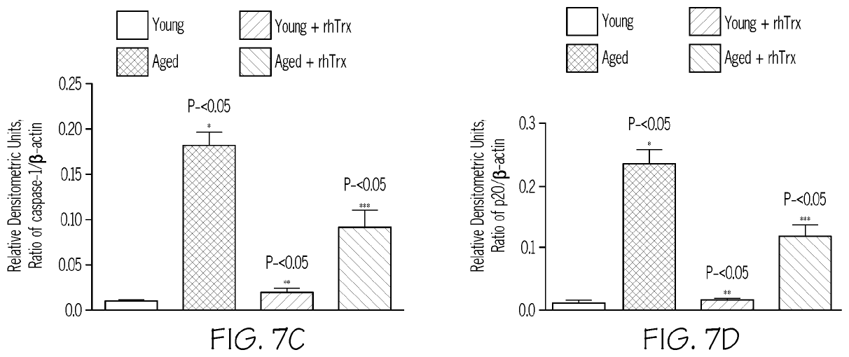

As shown in FIGS. 7A-7D, arteries from aged baboons showed increased NLRP3, caspase-1 and cleaved caspases p20 levels (FIGS. 7A-7D). However, baboons treated with rhTrx had significantly lower levels of NLRP3 and caspase-1/p20 activation (FIGS. 7A-7D).

Additionally, thioredoxin interacting protein (TXNIP) is known to activate inflammasome in oxidative stress due to oxidation of Trx. Therefore, Applicant hypothesize that rhTrx treatment will sequester TXNIP with rhTrx, thereby preventing its association with NLRP3 and resulting in failure of its activation in presence of rhTrx.

### Example 1.7. NLRC4 Expression is Increased in Arteries from Age Premenopausal Female Baboons, which is Decreased in rhTrx Treatment

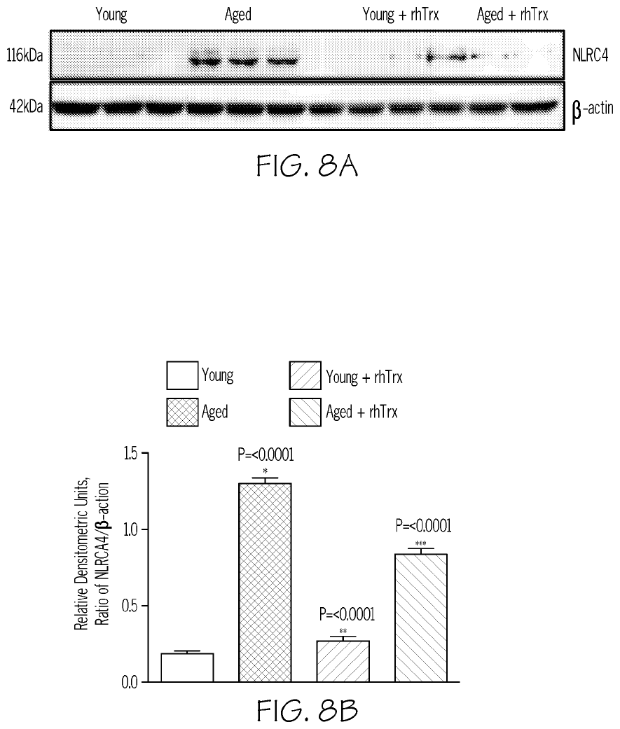

A recent study has shown increased expression of NLRC4 and IL-1b in humans with arterial stiffness. Applicant determined whether NLRC4 inflammasome expression is increased in arteries from aged baboon. As shown in FIGS. 8A-8B, NLRC4 protein expression is increased in the baboon arteries, which was decreased in arteries from rhTrx treated aged baboons, indicating NLRC4-mediated inflammation is an important contributor to arterial inflammation and consequent stiffness.

### Example 1.8. Recombinant Human Thioredoxin (rhTrx) Inhibits Transforming Growth Factor Beta (TGFb)-Mediated Increase in Collagen-I Expression and a-SMC Expression and Polymerization in Human Adventitial Fibroblasts (HAoAF)

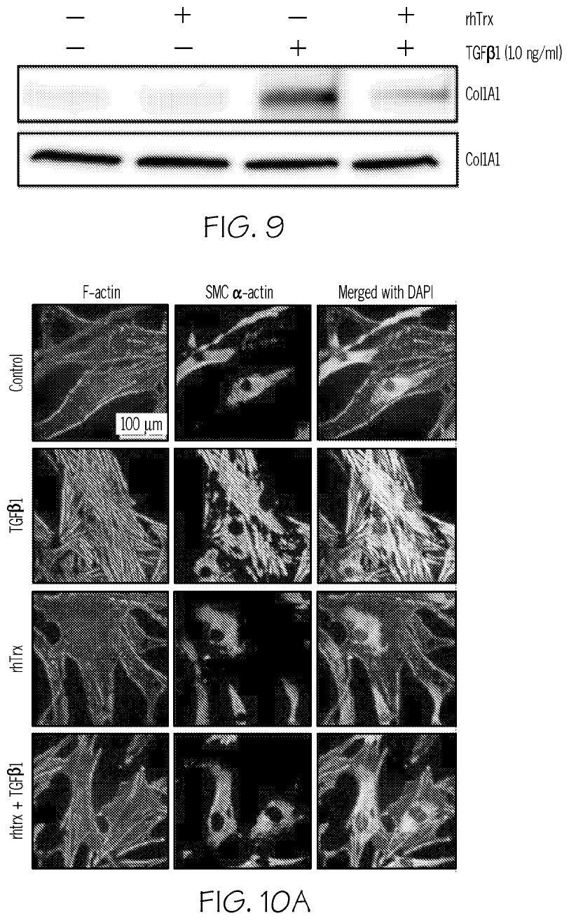

Next, Applicant determined whether rhTrx would directly inhibit TGFb-mediated signaling in human adventitial fibroblasts (HAoAF). As shown in FIG. 9, HAoAF showed increased Col1A1 expression when treated with TGFβ. However, treatment of cells with rhTrx significantly diminished Col1A1 expression.

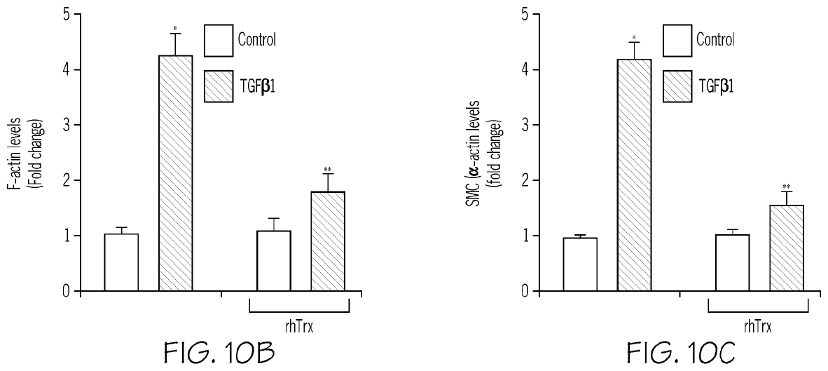

Applicant also determined whether rhTrx would impact transformation of fibroblasts to myofibroblasts by evaluating SMC actin polymerization. As shown in FIGS. 10A-10C, significant a-actin polymerization was observed in cells treated with TGFb, but rhTrx treatment significantly decreased actin polymerization (FIGS. 10B-10C), demonstrating efficacy of rhTrx in reducing vascular fibrosis.

### Example 1.9. Hydroxyproline Levels are Increased in Aortae of Aged Baboons but Decreased with rhTrx Treatment

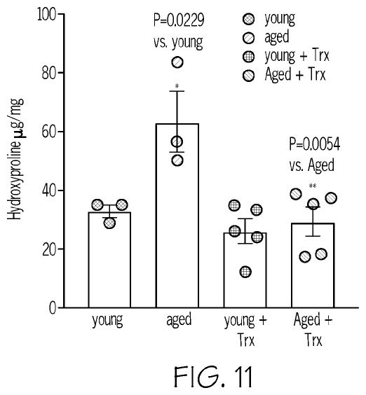

Hydroxyproline is the major amino acid in stiffer collagen fiber. Therefore, Applicant determined whether rhTrx would decrease hydroxyproline levels in aged arteries from baboons. As shown in FIG. 11, the level of hydroxyproline was significantly decreased in the aortae of aged baboons treated with rhTrx, suggesting that rhTrx treatment decreases collagen synthesis.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition for use in treating or preventing arterial stiffness in a subject, said composition comprising:
a recombinant human thioredoxin (rhTrx), derivatives thereof, peptide fragments thereof, mutants thereof, or combinations thereof.

2. The composition of claim 1, wherein the composition comprises recombinant human thioredoxin (rhTrx).

3. The composition of claim 1, wherein the composition comprises a peptide fragment of rhTrx.

4. The composition of claim 3, wherein the peptide fragment comprises SEQ ID NO: 2 or a sequence that shares at least 65% sequence identity to SEQ ID NO: 2.

5. The composition of claim 3, wherein the peptide fragment comprises SEQ ID NO: 3 or a sequence that shares at least 65% sequence identity to SEQ ID NO: 3.

6. The composition of claim 1, wherein the composition comprises at least 50 μM human recombinant thioredoxin.

7. The composition of claim 1, wherein the composition comprises at least 85 μM human recombinant thioredoxin.

8. The composition of claim 1, wherein the composition further comprises dithiothreitol (threo-1,4 Dimercapto-2,3-butanediol), ethylenediaminetetraacetic acid sodium, glycerol (1,2,3-Propanetriol), and an ammonium carbonate buffered solution (pH 7).

9. The composition of claim 1, wherein the composition comprises:
at least 85 μM human recombinant thioredoxin;
1 uM dithiothreitol (threo-1,4 Dimercapto-2,3-butanediol);
0.5 uM ethylenediaminetetraacetic acid sodium;
0.2% glycerol (1,2,3-Propanetriol); and
0.05 mM ammonium carbonate buffered solution.

10. The composition of claim 9, wherein the composition is administered at a dose of 250 μM to 500 μM of human recombinant thioredoxin in the composition of claim 9 per Kg of body weight of the subject.

11. A method of treating or preventing arterial stiffness in a subject, said method comprising:
administering a composition to the subject, wherein the composition comprises a recombinant human thioredoxin (rhTrx), derivatives thereof, peptide fragments thereof, mutants thereof, or combinations thereof.

12. The method of claim 11, wherein the composition comprises recombinant human thioredoxin (rhTrx).

13. The method of claim 11, wherein the composition comprises a peptide fragment of rhTrx.

14. The method of claim 13, wherein the peptide fragment comprises SEQ ID NO: 2 or a sequence that shares at least 65% sequence identity to SEQ ID NO: 2.

15. The method of claim 13, wherein the peptide fragment comprises SEQ ID NO: 3 or a sequence that shares at least 65% sequence identity to SEQ ID NO: 3.

16. The method of claim 11, wherein the composition comprises at least 50 μM human recombinant thioredoxin.

17. The method of claim 11, wherein the composition comprises at least 85 μM human recombinant thioredoxin.

18. The method of claim 11, wherein the composition further comprises dithiothreitol (threo-1,4 Dimercapto-2,3-butanediol), ethylenediaminetetraacetic acid sodium, glycerol (1,2,3-Propanetriol), and an ammonium carbonate buffered solution (pH 7).

19. The method of claim 18, wherein the composition comprises:
at least 85 μM human recombinant thioredoxin;
1 uM dithiothreitol (threo-1,4 Dimercapto-2,3-butanediol);
0.5 uM ethylenediaminetetraacetic acid sodium;
0.2% glycerol (1,2,3-Propanetriol); and
0.05 mM ammonium carbonate buffered solution.

20. The method of claim 19, wherein the composition is administered at a dose of 250 μM to 500 μM of human recombinant thioredoxin in the composition of claim 19 per Kg of body weight of the subject.

21. The method of claim 11, wherein the administration occurs by a method selected from the group consisting of intravenous administration, intramuscular administration, intradermal administration, intraperitoneal administration, subcutaneous administration, spray-based administration, aerosol-based administration, and combinations thereof.

22. The method of claim 11, wherein the administration occurs by intravenous administration.

23. The method of claim 11, wherein the subject is suffering from arterial stiffness.

24. The method of claim 11, wherein the subject is vulnerable to arterial stiffness.

25. The method of claim 11, wherein the subject is a human being.

26. The method of claim 11, wherein the arterial stiffness is associated with an arterial stiffness related disorder.

27. The method of claim 26, wherein the arterial stiffness related disorder is selected from the group consisting of cardiovascular disorders, cerebrovascular disorders, renal disorders, high blood pressure, hypertension, dementia, chronic kidney disorders, chronic vascular fibrotic disorders, vascular fibrosis, cardiac fibrosis, inflammation, chronic low-grade inflammation in aging, and combinations thereof.

28. The method of claim 26, wherein the arterial stiffness related disorder comprises a cardiovascular disorder.


SEQUENCE LISTING (3 sequences)

SEQ ID NO: 1 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 2 - protein, 60 aa, synthetic construct
KLVVVDFSATWCGPCKMIKPFFHSLSEKYSNVIFLEVDVDDCQDVASECEVKCMPTFQFF

SEQ ID NO: 3 - protein, 60 aa, synthetic construct
KLVVVDFSATWCGPCKMIKPFFHSLSEKYSNVIFLEVDVDDAQDVASEAEVKAMPTFQFF